(19) United States
(12) Patent Application Publication
(10) Pub. No.: US 2008/0140140 A1
(43) Pub. Date: Jun. 12, 2008

(54) Defibrillator Event Data with Time Correlation

(75) Inventors: Justin Grimley, Seattle, WA (US); Stephen LaBrash, Seattle, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/813,010

(22) PCT Filed: Dec. 29, 2005
(86) PCT No.: PCT/IB05/54425

§ 371 Date: Jun. 28, 2007

Related U.S. Application Data
(60) Provisional application No. 60/641,865, filed on Jan. 5, 2005.

(51) Int. Cl. A61N 1/00; A61B 5/02

(52) U.S. Cl. 607/5; 607/60; 600/481

(57) ABSTRACT

A defibrillator records event data with an elapsed time marker. Upon transfer of the event data to an event data recorder the elapsed time markers are used to time-stamp the event data with actual location time. The event date can be analyzed with a display of both elapsed time and actual location time.

This invention relates to defibrillators for ventricular fibrillation and, in particular, to an automatic external defibrillator (AED) for which transferred event data is time correlated to actual location time.

AEDs are in widespread use in public areas such as airports, shopping centers, and manufacturing and office buildings where they are available to respond to a person struck with sudden cardiac arrest characterized by ventricular fibrillation. Since AEDs are intended to be rushed to a patient and put into immediate use, they are generally not a.c. powered but are battery powered. And since AEDs spend most of their operational lives in readiness for a cardiac emergency it is important that their power consumption be kept low to extend the life of the battery. Usually only a blinking ready light and an occasional silent self-test are the only activities performed by an AED while it is awaiting use, thereby minimizing power consumption. Unlike a computer, there are minimal administrative tasks undertaken or supported by the AED. With regard to the present invention, AEDs generally do not support an onboard real time clock or calendar which needs to be maintained and reset each time the battery is replaced, for instance. Instead, there is a basic time counter that simply counts the elapsed time since the battery was last inserted into the AED. When the AED is put to use in a cardiac emergency the activities of the AED (event data) can be recorded together with the elapsed time counts of the counter. However, following a handoff of a patient to professional medical personnel from a first responder, or at later times when data of the response is being reviewed, it is desirable to correlate the event data to the actual clock and calendar times when the events of the event data occurred. One approach to providing this capability is a data card that can be unplugged from the AED and transferred to a data recording or analysis system as shown in U.S. Pat. No. 5,549,115. This data card has a real time clock maintained on the card and consequently is able to convey real time clock data along with the event data to an analysis or recording system. See also U.S. Pat. Nos. 5,680,864 and 5,785,043. However, in order to support the real time clock while the data card is being transferred between devices the card requires an onboard battery. Thus, it is necessary to maintain such a battery so that the data cards are always operable and will not lose the real time data. Accordingly it is desirable to provide a technique for transferring event data with real time data without the cost or upkeep of a battery-powered data card and without the need to support and update a real time clock on the AED.

In accordance with the principles of the present invention, an AED is provided which transfers cardiac event data to a recording or analysis device. The AED has an onboard elapsed time counter which is used to mark event data recorded on the AED. The count of the elapsed time counter is transferred along with the transfer of event data and is used to correlate the elapsed time markers of the event data to actual location time on the recording or analysis device. In an illustrated embodiment the event data is subsequently displayed with both its elapsed time and actual location time information.

In the drawings:

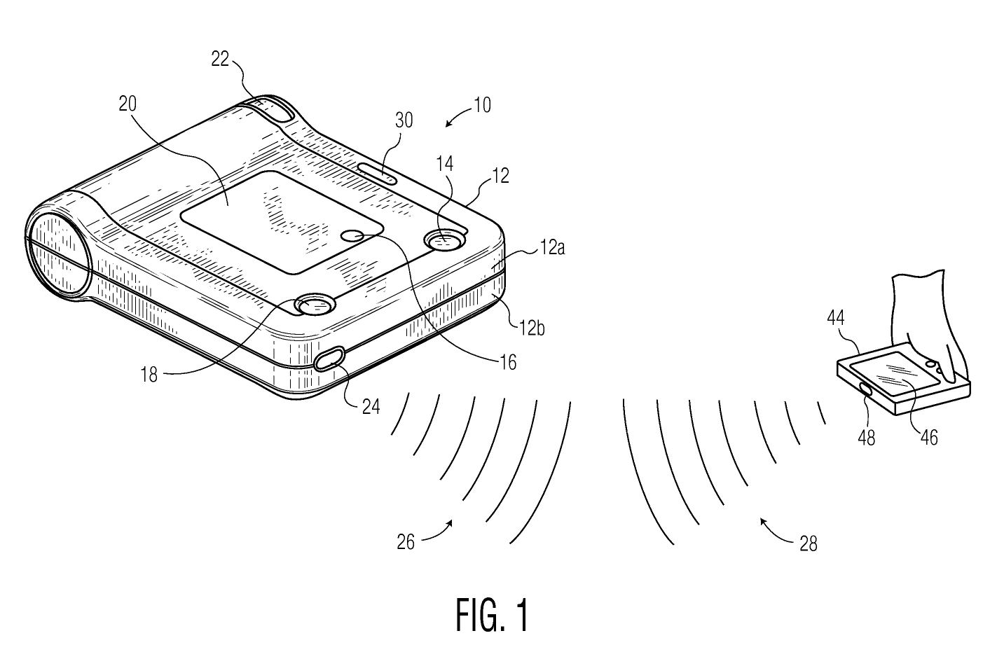

FIG. 1 illustrates the transmission of AED event data from an AED to an event data recorder;

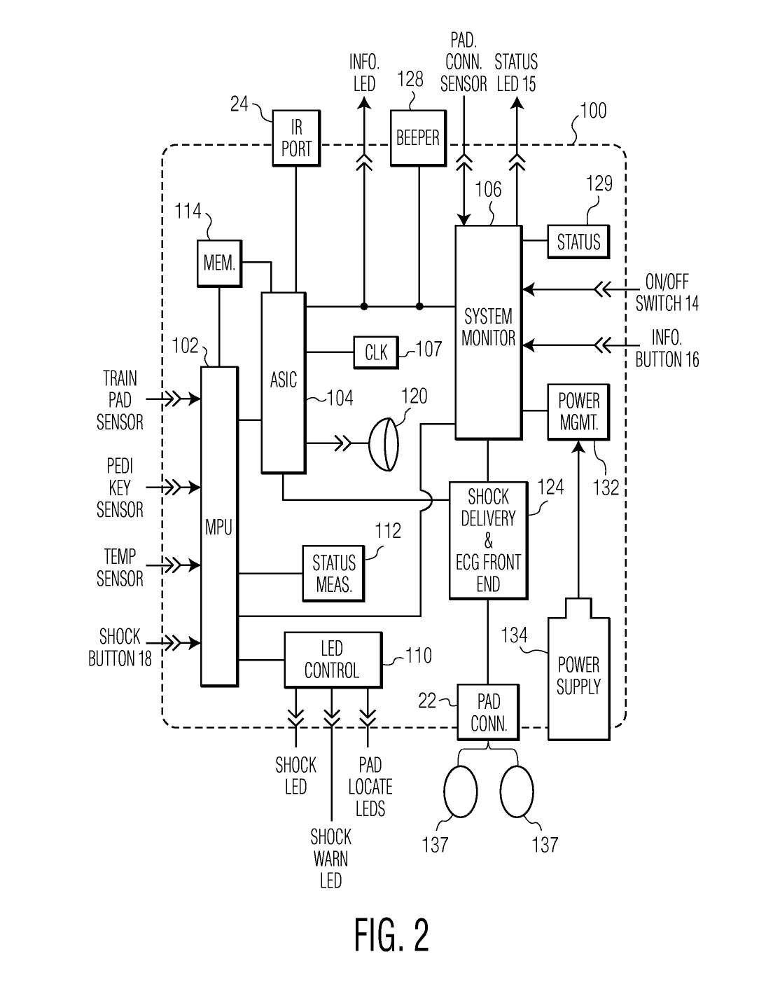

FIG. 2 illustrates in schematic diagram form the major operational subsystems of an AED;

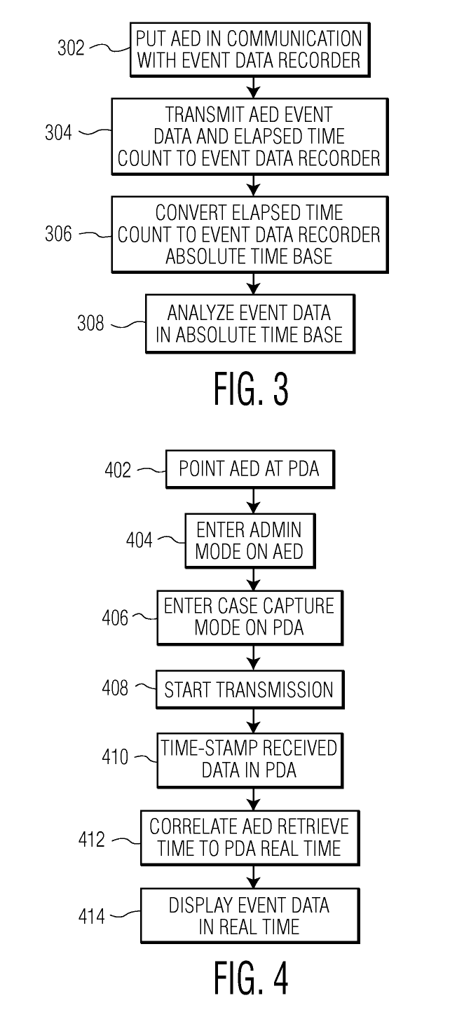

FIG. 3 is a flowchart of a first embodiment of the present invention;

FIG. 4 is a flowchart of a second embodiment of the present invention;

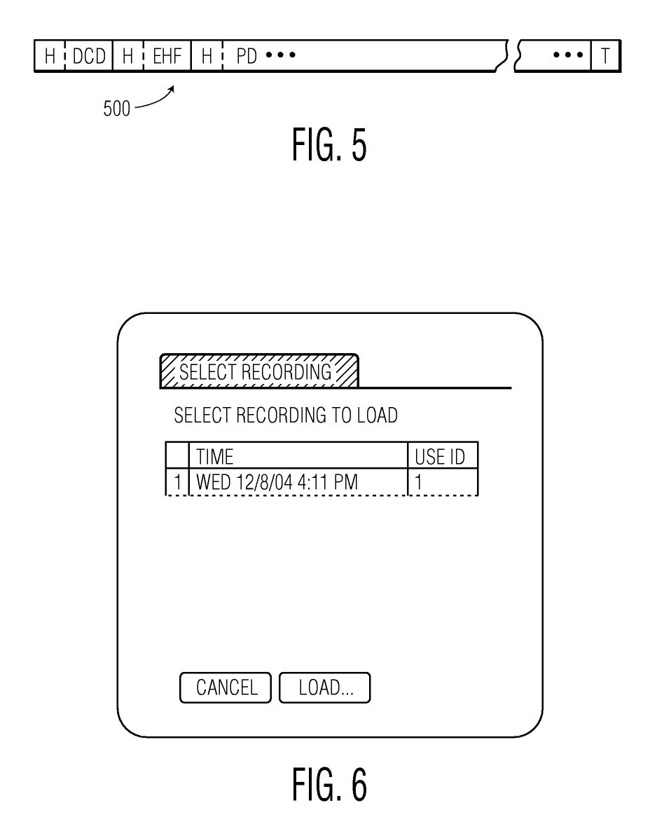

FIG. 5 illustrates a data format by which event data can be transmitted from an AED to a recording or analysis device;

FIGS. 6-9 illustrate display screens of an event data recorder in accordance with the principles of the present invention; and

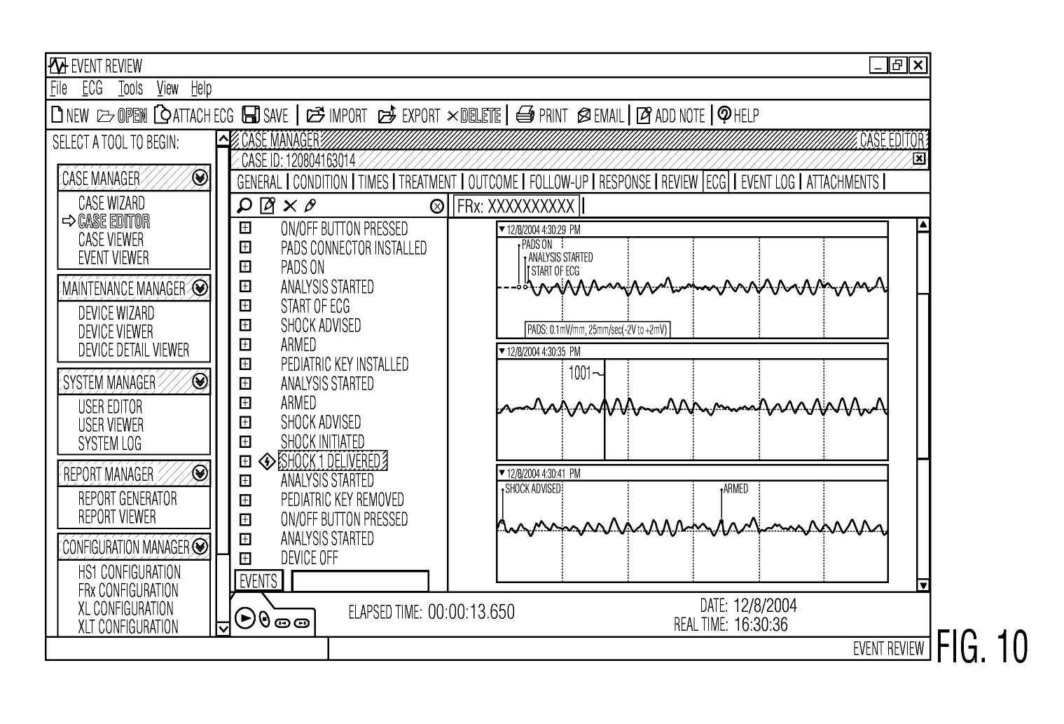

FIG. 10 illustrates a display screen of an analysis device in accordance with the principles of the present invention.

Referring first to FIG. 1, an AED unit 10 is shown in perspective. The AED unit is enclosed in a case 12 having two halves 12a and 12b which are sealed together to prevent the ingress of water, dust and contaminants to the electronic modules inside the case. The AED unit 10 has an on/off button 14 and a shock button 18 which is depressed to deliver a defibrillating shock. The AED unit has an information button 16 which flashes when information concerning a protocol is available for the operator. The AED unit has a panel 20 which is a display for the display of visual information for the operator such as the proper placement of electrode pads on the patient. In this embodiment information and instructions are delivered audibly through a loudspeaker or headset. The AED unit has a connector 22 into which the mating connector of an electrode pad set is plugged. At the top of the case is a slot 30 into which a key-like device is inserted to switch the AED to a pediatric rescue protocol. An infrared port 24 for data communication to and from the AED unit is located on the side of the case 12. The waves 26 indicate the transmission of infrared signals to and from the AED 10.

In accordance with the principles of the present invention the AED unit 10 transmits event data through the infrared port 24 which is received by an event data recorder 44. The event data recorder 44 is a small digital device such as a personal digital assistant (PDA) with an infrared communication port 48 which can send and receive encoded infrared waves 28. The event data recorder communicates with the AED with a handshake protocol, first transmitting information such as a request for data, which is responded to by a transmission of data from the AED which is received by the event data recorder. The event data recorder 44 has a screen 46 which provides a user interface for executing a transfer of data and on which the operator can see an identification of the event data received from the AED as described more fully below.

The major components of an AED are shown in FIG. 2 in block diagram form. Further detailed information about the operation of an AED can be obtained in U.S. Pat. No. 5,836,993, to Cole for “Electrotherapy Device Control System and Method,” the specification of which is incorporated herein. In the illustration of FIG. 2, defibrillator control functions are divided among a microprocessor unit (MPU) 102, an application-specific integrated circuit (ASIC) 104 and a system monitor 106.

MPU 102 performs program steps according to software instructions provided to it from memory 114 which may comprise one or more of EPROM, RAM and flash ROM memory. The MPU also stores status data concerning the AED in the memory 114 as well as samples of patient data, e.g., ECG waveform samples, acquired during use of the AED. MPU 102 controls the operation of certain system LEDs through an LED control circuit 110, including an LED associated with the shock button 18, the LED associated with a Do Not Touch indicator, and LEDs which indicate the proper placement of the electrode pads on the torso of the patient. MPU 102 also receives system status information as shown by block 112, temperature information from the interior of the case 12 from a temperature sensor (not shown), and a signal from a sensor when training pads are plugged into the connector 22. The training pad sensor can be a magnetic sensor associated with connector 22 which senses the field of a small magnet integrated into the connector of a training electrode pad set. The MPU is also responsive to a signal from a sensor associated with slot 30 when a key is inserted into the slot to switch the operation of the AED unit to a pediatric rescue protocol.

ASIC 104 implements the memory map to system memory 114. ASIC 104 is clocked by a clock 107 and also controls a speaker 120 which delivers audible instructions during use of the AED. ASIC 104 can actuate a relay within the shock delivery and ECG front-end system 124 in response to actuation of the shock button 18 by a user during treatment. ASIC 104 will actuate an LED associated with the information button to signal to the user that information is available and can be accessed by depressing the information button 16. The ASIC also provides the interface to the IR port 24 through which new program information can be loaded into the AED unit and event data can be communicated to a separate data storage or analysis system. The ASIC 104 can also provide the interface to a display at panel 20 when the AED is to be equipped with a display for AED and patient information.

System monitor 106 performs automatic self-tests of the AED and its components. The system monitor 106 controls the status LED 15 to indicate that the self-tests are showing proper system operation, and activates beeper 128 to provide an audible alert when the system is not operating properly. Details of suitable self-tests may be found in U.S. Pat. No. 5,879,374, to Powers, et al. for “External Defibrillator with Automated Self-Testing Prior to Use,” the specification of which is incorporated herein by reference. System monitor 106 is also the defibrillator's interface with the on/off switch 14, the information button 16, and a sensor associated with connector 22 which signals the connection of a specific type of electrode pads 137 to the AED unit. System monitor 106 controls a power management subsystem 132 to provide power to operate system components from a removable battery 134 and to provide energy to the shock delivery system's capacitor(s) for a therapeutic shock during treatment. System monitor 106 also interfaces with the defibrillator's ECG front end, enables the shock delivery system to deliver a shock in response to detection of a patient ECG pattern requiring treatment (and actuation of the shock button 18), and controls delivery of the shock to electrode pad connector 22 and electrode pads 137 in response to shock delivery status information (e.g., patient impedance) obtained during delivery of the shock. Further information regarding this last function may be found in U.S. Pat. No. 5,735,879 to Gliner et al. for “Electrotherapy Method for External Defibrillators,” and U.S. Pat. No. 5,607,454, to Cameron et al. for “Electrotherapy Method and Apparatus,” the specifications of which are incorporated herein.

These defibrillator components communicate with each other over suitable communication buses, as shown in FIG. 2.

The system monitor 106 includes a counter which begins counting from zero each time a charged battery is inserted into the AED. This counter records the elapsed time in seconds since the battery was inserted. When the AED is in operation to treat a patient, data which is stored in memory 119 concerning the treatment event is tagged with the count of the elapsed time counter. For example, when the on/off button 14 is depressed to turn on the AED, the AED status of power-on may be recorded in the memory along with the time at which it occurred, for example, a count of 2,592,000 which means that 2,592,000 seconds have elapsed between the time the battery was installed in the AED and the time of power-on. When the AED is powered up to treat a patient another event which is time-stamped is the deployment of the electrode pads on the patient. The patient's ECG waveform is sampled and recorded and the stored samples are time-stamped. The decision to shock is recorded and time-stamped, as are parameters of the shock including patient impedance, voltage and shock duration. All of the times are recorded in elapsed time from battery installation.

At the time of the rescue by a layperson first responder or immediately thereafter an emergency medical responder is contacted to take over the treatment of the patient. The medical responder may continue to leave the patient connected to and monitored by the AED, but generally the medical responder will unplug the electrode pads from the AED and then connect the electrode pads to a higher performance unit such as the HeartStart MRx monitor/defibrillator from Philips Medical Systems, which can continue to monitor the patient's vital signs and apply further shocks if needed.

With the patient now transferred to the care of a medical professional, the medical responder will want to take the AED event data to the hospital or trauma center where the patient will receive further care and treatment so that the event data can be reviewed by the treating physician. In accordance with the principles of the present invention the AED event data is transferred to an event data recorder 44 as shown by the flowchart of FIG. 3. The first step 302 is to put the AED 10 into communication with the event data recorder 44. This can be done by connecting the event data recorder to the AED with a data cable and transmitting the event data over a cable. In the embodiment shown in FIG. 1 the event data is transmitted wirelessly by an infrared communication link. Other wireless transmission techniques such as “Wy-fi” may alternately be employed. When the wireless link is a line-of-sight link such as the optical one depicted in FIG. 1, the two units are put into communication with each other simply by directing the infrared port of the event data recorder at the infrared port of the AED. The event data and its markers of the elapsed time counts are then transmitted in step 304 from the AED to the event data recorder. In step 306 the elapsed time counts received by the event data recorder are converted to the absolute time base of the event data recorder, which is the actual time and date at the location of the incident. This is done by correlating the absolute time base to the elapsed time count at the time of the data transfer, then using the elapsed time counts of the transferred data to measure back in time for the absolute times of the event data. The final step in the sequence is to analyze the event data in the absolute time base, examples of which are given below.

FIG. 4 illustrates another embodiment of the present invention. In this example the event data recorder is a PDA which contains software to conduct a transfer of data with an AED. This transfer software has a “Case Capture Mode” which is the mode in which a wireless data transfer is conducted. The procedure begins at 402 by pointing the AED's infrared port (in the case of an infrared communication) at the infrared port of the PDA. The operator enters the “Administrative Mode” on the AED at 404, the mode in which a data transfer is conducted. Correspondingly, the operator enters the Case Capture Mode on the PDA in 406. Data transmission between the AED and the PDA then begins at 408.

In this example the data transmission is commenced by the PDA, which sends a data request to the AED. The transmission continues in this handshake fashion, with a request from the PDA followed by a response of data from the AED. A typical format for the data is shown in FIG. 5. In this example the files exchanged include data packaged into records or events which use a record header denoted by “H” in the data sequence 500 of FIG. 5. Each header H contains a time marker which is the elapsed time in seconds since the last battery insertion at which the data was recorded. In this sequence the first file is a Device Configuration Data file (DCD) which contains data on the setup configuration of the AED. This is followed by an Event History file (PUD) which is a listing of all of the rescue episodes or event recordings currently stored on the AED. In this embodiment all events stored on the AED are transferred when a transfer is started, to relieve the operator of the need to find and assure the transmission of a particular set of event data. An entry appears in the Event History file for each patient event which is transferred to the event data recorder. In the illustrated sequence 500 the transmission of the Event History file is followed by a transmission of the Patient Data (event or “use” data) which consists of data such as impedance data, common-mode current data and electrocardiogram (ECG) data. Other files such as test data files may also be transmitted. At the end of the illustrated sequence is a trailer “T” which contains a time marker and marks the end of the data transmission.

At 410 the data received by the PDA is time-stamped at least once with the time reference of the PDA to correlate the elapsed time data of the AED with the time base of the PDA. In this example the time base of the PDA is the actual local time and date at the location of the PDA. For example, this time-stamping can be done with the elapsed time data of the first header received by the PDA. For instance, this time-stamping might correlate an elapsed time count of 2,593,800 seconds with the time/date reference of the PDA of 4:31 pm on Dec. 8, 2004. The actual time of the power-on event, previously illustrated as occurring at an elapsed time of 2,592,000, can now be computed to have occurred at 1800 seconds earlier, which is 4:01 pm on Dec. 8, 2004. The other elapsed time markers in the data stream can be similarly correlated as indicated at 412. In this example the time-stamping occurs at the initial elapsed time marker in the header at the beginning of the transmission and at the last elapsed time marker in the trailer at the end of the transmission. The time interval between these two markers can be used to more precisely time-stamp the intervening event data later. In a constructed embodiment it may be desirable to time-stamp every elapsed time marker at the time it is received, thereby correlating all times in the transmission by the end of the transmission.

At 414 the event data is displayed in relation to the actual local time. One way this may be done is by a display of a patient use directory on the screen of the PDA as shown in FIG. 6. The patient use directory may be formatted in the AED prior to transmission to the PDA, or the Event History file data may be parsed into individual uses for the displayed directory after the Event History file has been received by the PDA. In this example the AED contained data from only a single patient event or use, identified on the screen as Use ID 1. The patient use directory shows that this event or use occurred on Wednesday, Dec. 8, 2004 at 4:11 pm.

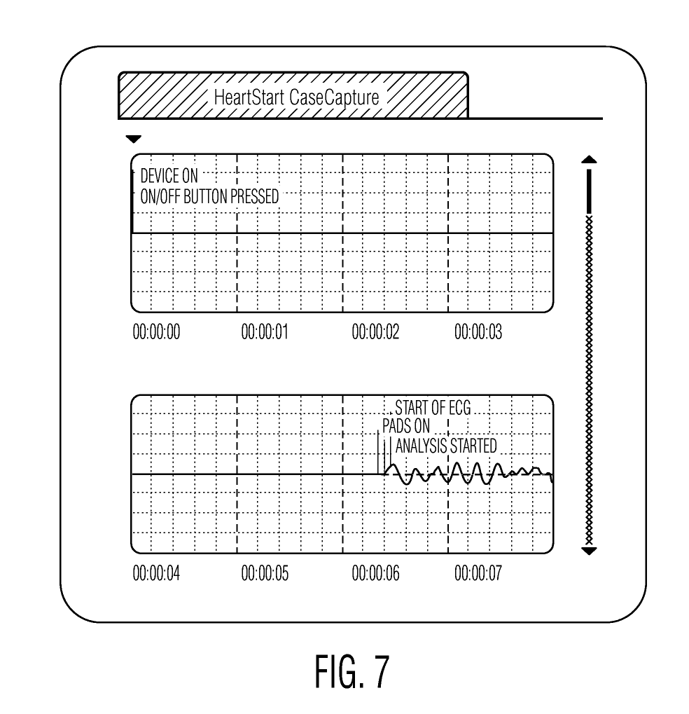

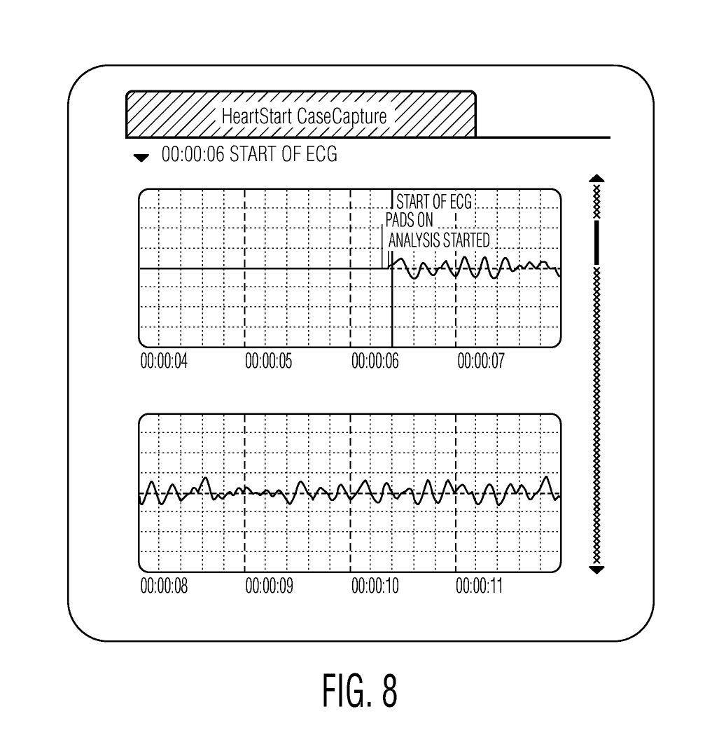

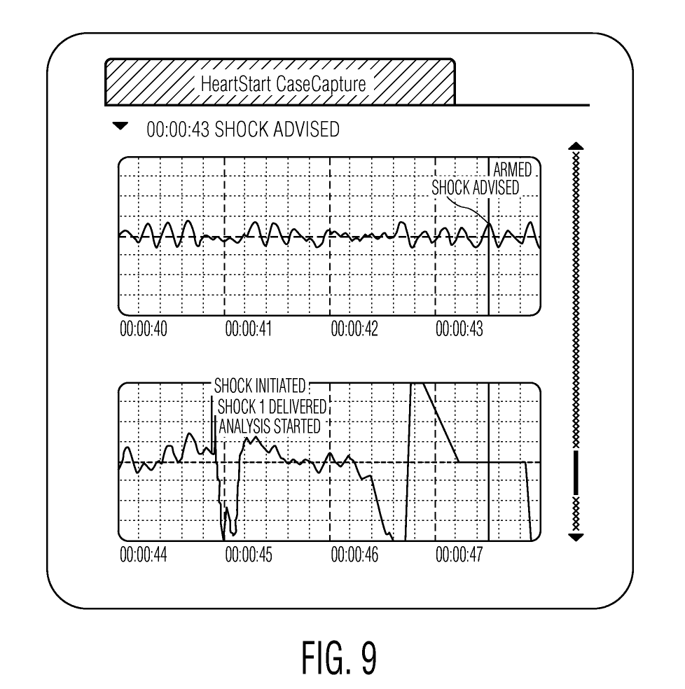

In accordance with a further aspect of the present invention the attending physician can use the PDA to do a limited review of the event data stored in the PDA in the emergency room where the patient is transported, for instance. The physician calls up the Select Recording screen shown in FIG. 6 and selects one of the events in the patient use directory with a wand or pointer. The physician then selects “Load”, and the selected event data is loaded in the PDA for display as illustrated in FIGS. 7-9. FIG. 7 shows the ECG data starting from the time the AED was powered on against an elapsed time scale which references the power-on time as time 00:00:00. In this example six seconds pass at which point the electrode pads are attached to the patient, an ECG waveform is detected and the waveform is analyzed. The physician can slide the display bar on the right side of the screen to view later segments of the ECG data such as the next segment shown in FIG. 8. The physician is also given the choice of pulling down the menu at the upper left of the screen where major occurrences in the event data are listed. FIG. 8 shows one such occurrence, the Start of ECG, which is shown as occurring at 6 seconds following power-on. The physician could select Shock Advised from this pull-down menu and the display will jump to the segment where this event occurred as shown in FIG. 9, where a shock was advised 43 seconds after power-on. The major occurrences in the event data are marked with colored vertical lines on the ECG display and are also annotated as illustrated in FIGS. 8 and 9.

When the physician wants to review and analyze the event data the data is transferred from the event data recorder 44 to a personal computer or workstation (PC). This can be done by transferring the data to the PC by means of a cable or the infrared or other wireless link. When a PDA is used as the event data recorder the data can conveniently be transferred by placing the PDA in its docking cradle connected to the PC and the data transferred as a part of or in addition to a synchronization of the PDA. During the synchronization the real time date/clock of the PDA and the PC can be brought into synchronization so that the PC display will show the events accurately time-stamped. FIG. 10 illustrates a typical PC display of event data called “Case Manager.” In this embodiment the functions of the Case Manager are shown in the left-hand column. On the Case Manager display screen segments of the ECG event data are shown, with the major occurrences listed to the left of the ECG data. The user can click on one of the listed major occurrences and the data display will show the segment of the ECG trace which contains that occurrence. The major occurrences are marked with a colored vertical line and a textual annotation as they were on the PDA display. In addition, a differently colored vertical line 1001 is shown in the middle strip of the ECG display which scrolls through the ECG data. The date and time of the beginning of each strip is shown to the upper left of each strip, converted into actual location time from the elapsed time data recorded by the AED. The date and time at the point of the vertical line 1001 is shown at the bottom of the screen in two ways. In addition to the date of the ECG data, Dec. 8, 2004 in this example, the time of the scrolling vertical line 1001 is shown in both elapsed time and in actual location time. In this example the elapsed time at the point of the line 1001 is 13.65 seconds from power-on of the AED, which is 16:30:36 in “real time” (location time). If the reviewer scrolls or moves the line 1001 to a different point on the display the elapsed time and real time displays at the bottom of the screen are updated accordingly.

1. A defibrillator system including a defibrillator which analyzes a heartbeat and determines whether a defibrillating shock is to be delivered, including an ECG data analyzer and a high voltage shock delivery circuit comprising:
a data storage medium which stores data concerning operation of the defibrillator together with elapsed time data associated with the operation data; and
a wireless transmitter, coupled to the data storage medium, by which the operation data and time data are transmitted; and
an event data recorder, including a wireless receiver, which executes a control program for the reception and storage of the transmitted operation data and time data, the event data recorder including a converter operable to convert the elapsed time data to an absolute time base of the event data recorder.

2. The defibrillator system of claim 1, wherein the operation data comprises patient event data obtained during treatment of a patient;
wherein the time data comprises elapsed time data associated with the patient event data; and
wherein the event data recorder converter further comprises a correlation processor which correlates the elapsed time data with a time base of the event data recorder.

3. The defibrillator system of claim 2, wherein the time base of the event data recorder comprises actual time at the location of the event data recorder.

4. The defibrillator system of claim 2, wherein the correlation processor correlates at least one piece of elapsed time data during or promptly following transmission of the event and elapsed time data by the defibrillator transmitter.

5. The defibrillator system of claim 4, wherein the correlation processor correlates one piece of elapsed time data at or near the beginning of the transmission, and correlates a second piece of elapsed time data at or near the end of the transmission.

6. The defibrillator system of claim 2, wherein the event data recorder further includes a display which is operable to display information concerning received event data and its associated time in the time base of the event data recorder.

7. The defibrillator system of claim 1, wherein the event data recorder comprises a personal digital assistant.

8. A defibrillator system including a defibrillator which analyzes a heartbeat and determines whether a defibrillating shock is to be delivered, including an ECG data analyzer and a high voltage shock delivery circuit, the defibrillator further including a data storage medium which stores data concerning operation of the defibrillator together with time data associated with the operation data; and
an event data recorder which is responsive to the reception of operation data and time data from the defibrillator, the event data recorder including a time-stamp responsive to the time data for time-stamping received operation data to the time base of the event data recorder; and
a communication link, which is operable to communicate data between the defibrillator and the event data recorder.

9. The defibrillator system of claim 8, wherein the defibrillator time data comprises elapsed time data and wherein the event data recorder time base comprises real time at the location of the event data recorder.

10. The defibrillator system of claim 9, wherein the event data recorder time stamp acts to time stamp operation data received from the defibrillator in real time.

11. The defibrillator system of claim 10, wherein the event data recorder further comprises a display which is operable to display operation data received from the defibrillator and associated real time.

12. The defibrillator system of claim 11, wherein the display is further operable to control the communication link.

13. The defibrillator system of claim 9, further comprising an event analysis computer, responsive to operation data received from the defibrillator, which acts to display the operation data in both elapsed time and in real time.

14. The defibrillator system of claim 8, wherein the event data recorder includes a display which is operable to display operation data of the defibrillator in both elapsed time and in real time.

15. A method for correlating cardiac event data stored by an AED to an absolute time base comprising:
putting the AED in communication with an event data recorder;
transmitting event data and associated elapsed time data from the AED to the event data recorder;
converting at least one piece of the elapsed time data to an absolute time base in the event data recorder; and
displaying the event data with associated absolute time data.

16. The method of claim 15, wherein converting further comprises converting one piece of elapsed time data at the beginning of the transmission and another piece of elapsed time data at the end of the transmission to the absolute time base.

17. The method of claim 15, wherein putting the AED in communication with an event data recorder comprises establishing an infrared data link between the AED and the event data recorder.

18. The method of claim 15, further comprising coupling the event data from the event data recorder to an analysis computer;
wherein displaying the event data with associated absolute time data comprises displaying the event data and associated real time on the analysis computer.

19. The method of claim 18, wherein displaying the event data and associated real time on the analysis computer further comprises displaying elapsed time associated with the event data on the analysis computer.
